(19) United States
(12) Patent Application Publication
(10) Pub. No.: US 2020/0222577 A1
(43) Pub. Date: Jul. 16, 2020

(54) BIOMATERIALS COMPRISING GELATIN DERIVED FROM COLD-ADAPTED AQUATIC SPECIES

(71) Applicants: UNIVERSIDAD DE LOS ANDES, Las Condes Santiago (CL); UNIVERSIDAD TÉCNICA FEDERICO SANTA MARÍA, Valparaíso (CL)

(72) Inventors: Javier ENRIONE CÁCERES, Las Condes Santiago (CL); Cristian Andrés ACEVEDO GUTIÉRREZ, Valparaiso (CL); Elizabeth Yeny SÁNCHEZ MONTIEL, Valparaíso (CL)

(21) Appl. No.: 16/627,242

(22) PCT Filed: Jun. 29, 2018
(86) PCT No.: PCT/IB2018/054866

§ 371 Date: Dec. 27, 2019

(30) Foreign Application Priority Data
Jun. 30, 2017 (EP) 17179213.8

(51) Int. Cl. A61L 15/32; A61L 15/20; A61L 27/22; A61L 27/26

(52) U.S. Cl. CPC A61L 15/32; A61L 27/26; A61L 27/222; A61L 15/20

(57) ABSTRACT

The present document describes a composition and pharmaceutical composition comprising gelatin derived from a cold-adapted aquatic species, chitosan, agarose and glycerol. Further, the present document discusses a process for manufacturing a biomaterial which comprises gelatin derived from a cold-adapted aquatic species, chitosan, agarose and glycerol. The biomaterial obtained or obtainable through the process is also described. Also, the present document describes a kit which comprises: gelatin derived from a cold-adapted aquatic species, chitosan, agarose, and glycerol. Lastly, the use of the composition, pharmaceutical composition, biomaterial or kit for the production of scaffolds, dressings, beads, engineered tissues, devices or micro-devices suitable for therapeutic or diagnostic purposes or the use of the composition, biomaterial or kit for tissue engineering are also discussed.

### TECHNICAL FIELD

The present invention relates to the field of medicine. In particular, the present invention provides a composition, pharmaceutical composition or kit comprising a gelatin polymer derived from a cold-adapted aquatic species, a process to manufacture a biomaterial comprising a gelatin polymer derived from a cold-adapted aquatic species and the use of the composition, pharmaceutical composition, biomaterial or kit for certain applications.

### BACKGROUND ART

Hydrogels are semi-solid structures which comprise networks of insoluble polymers surrounded by water (Lee, et al., 2001. “Hydrogels for Tissue Engineering,” Chem. Rev., 101(7): 1869-1880). Hydrogels are attractive materials for tissue engineering. Particularly attractive are those materials which can be polymerized in aqueous solutions, which can be injected or placed on wounds and other defects and which form a stable matrix for cell growth, remodeling and tissue regeneration.

Most sources of commercial gelatin are derived from mammalian tissue (e.g. muscles, skin, tendons, etc.). Gelatin is a proteic material which functions as a support for tissues. Gelatin is biocompatible, biodegradable, and possesses little or no antigenicity. The structural conformation of gelatin in water is temperature-dependent; at above 30° C., the gelatin is in a disordered conformation, random coil and at temperatures below 25° C. it forms a semi-ordered network of triple helices with a molecular weight of about 300 kDa. The temperature at which gelatin transitions from a solution to a gel is known as the gelation temperature (T_{G}).

The kinetics of triple helices formation, the T_{G} and the structural stability of the triple helices are dependent on the amino acid composition of the gelatin, especially on the proportion of proline and hydroxyproline. Another structural characteristic of gelatin is the RGD (Arg-Gly-Asp) motif which favors cellular adhesion and tissue regeneration.

For the above reasons, gelatin has advantages over other biomaterials that are used in tissue engineering. Since gelatin produces biocompatible materials which are also biologically inert, a lot of research has focused on the creation of biomaterials for regenerative medicine.

Gelatin derived from salmon skin also contains the RGD motif. However, salmon skin-derived gelatin has different viscoelastic properties than mammalian-derived gelatin and it has a T_{G} of around <10° C. Once the salmon skin gelatin (SG) forms a stable triple helices structure it is less stable than mammalian gelatin because the SG has a lower molecular weight distribution (around 150 kDa) and a lower proportion of proline and hydroxyproline.

A composition comprising collagen or gelatin, one or more crosslinkers and a plasticizer for use as a tissue sealant has been described in WO 97/29715. In particular, WO 97/29715 discloses the use of aldehydes as crosslinkers.

US 2002/0015724 describes a composition comprising type I and/or III polymerized collagen and a composition comprising gelatin for use as a sealant or as a dressing. In particular, monomeric collagen is produced and polymerized using an adequate reagent.

U.S. Pat. No. 6,007,613 discloses a biological adhesive comprising two components. The first component is a solution comprising gelatin and the second component, which may be in a gel-like state, comprises an aldehyde.

WO 2006/083384 describes a tissue adhesive which is prepared by crosslinking albumin and/or gelatin with specific polyamines and/or polycarboxylates using soluble carbodiimide.

There is currently a need for a composition or biomaterial which is biocompatible for use in the field of regenerative medicine. The present invention provides a composition or biomaterial which can be used in the field of regenerative medicine.

### FIGURES

FIG. 1: Image of the biomaterial of the present invention. The biomaterial was sponge-like and homogenous.

FIG. 2: Scanning electron microscopy images of the material obtained at the different stages of biomaterial manufacture. The images are of 150× or 500× magnification. The stage 1 material, which is obtained after lyophilizing the initial mixture of components, produces a porous material. The stage 2 material, i.e. after crosslinking, has a more organized and stable porous structure. The porous structure of the stage 2 material appears to be unaffected by gamma radiation as can be seen in the images of the stage 3 material.

FIG. 3: Dynamic vapor sorption of the materials obtained at the different stages of biomaterial manufacture. All the materials appear to interact similarly with water.

FIG. 4: Differential scanning calorimetry (DSC) of the materials obtained at the different stages of biomaterial manufacture. A) Representative DSC melting profiles of a single sample. B) Graph showing the glass transition temperature (Tg) of the materials obtained at the different stages. Data points represent the mean±1 standard deviation (n=3). C) Graph showing the heat capacity (Cp) of the materials obtained at the different stages. Data points represent the mean±1 standard deviation (n=3). D) Graph showing the melting temperature (Tm) of the materials obtained at the different stages. Data points represent the mean±1 standard deviation (n=3). E) Graph showing the change in enthalpy (AH) of the materials obtained at the different stages. Data points represent the mean±1 standard deviation (n=3). ETAPA 1=STAGE 1. ETAPA 2=STAGE 2. ETAPA 3=STAGE 3.

FIG. 5: In vivo study on the biocompatibility, biosecurity and biodegradability of the sterilized biomaterial. A) Growth curves of the rabbits wounded and then exposed to a dressing made of the sterilized biomaterial produced in Example 2. B) Images of the wounds taken at different points during the recovery of the rabbits.

FIG. 6: Photomicrographs of histological sections. A-F: Panoramic view showing the entire scar and normal skin edges with hair follicles (hf). Except the rabbit A, where a small area without epithelium (arrow) persists underlying remnants of scab (Sc); in all areas of implant for each rabbit there is a continuous and thick epidermis (asterisk). This layer of epidermis of greater or lesser extension (arrowheads demarcate the area), is covering the dermis of connective granulation tissue (GT) typical of the healing process. Scale bar=1 mm A′-F′: to further increase photomicrographs showing the epidermal tissue hyperplastic (asterisk) and its epidermal ridges (C′-F′, double arrows) projecting into the dermis, in areas where the dermis is observed more lax (CT). In the basal epithelial layer resting on a blue basal lamina, cells in mitosis can be observed (A′; anaphase, thick arrow). Scale bar=50 μm.

### SUMMARY OF THE INVENTION

The present invention provides a composition and a pharmaceutical composition comprising gelatin derived from a cold-adapted aquatic species, chitosan, agarose and glycerol. Further, the present invention provides a process for manufacturing a biomaterial which comprises the steps of: a) mixing gelatin derived from a cold-adapted aquatic species with chitosan, agarose and glycerol; b) drying the solution obtained in step (a); and c) chemically crosslinking the mixture of step (b). The biomaterial obtained or obtainable through the process is also an aspect of the present invention. Also, the present invention provides a kit which comprises: (i) gelatin derived from a cold-adapted aquatic species; (ii) chitosan; (iii) agarose; and (iv) glycerol. Lastly, the use of the composition, biomaterial or kit of the present invention for the production of scaffolds, dressings, beads, engineered tissues, devices or micro-devices suitable for therapeutic or diagnostic purposes or the use of the composition, biomaterial or kit of the present invention for tissue engineering are also aspects of the present invention.

### DETAILED DESCRIPTION OF THE INVENTION

### Definitions

The term “gelatin” refers to a hydrolyzed form of collagen, wherein the hydrolysis results in the reduction of the protein fibrils into its constituent polymer chains.

The term “cold-adapted aquatic species” refers to any cold-blooded organism which has evolved to reside in an aquatic environment where the temperature of the environment is cold, preferably 15° C. or less. In a preferred embodiment, the organism is a vertebrate.

The term “chitosan” refers to a linear polysaccharide composed of randomly distributed β-(1→4) D-glucosamine and N-acetyl-D-glucosamine. It is obtained by treating chitin with an alkaline substance.

The term “agarose” refers to a linear polymer made up of a repeating unit called agarobiose (a disaccharide made up of D-galactose and 3,6-anhydro-L-galactopyranose) which can be extracted from seaweed.

The terms “individual”, “patient” or “subject” are used interchangeably in the present application and are not meant to be limiting in any way. The “individual”, “patient” or “subject” can be of any age, sex and physical condition.

The term “wound” refers to any injury to living tissue caused by a cut, blow or other impact.

### Composition

In a first aspect, the present invention provides a composition comprising: (i) 0.3 to 2% (w/v) gelatin derived from a cold-adapted aquatic species; (ii) chitosan; (iii) agarose; and (iv) glycerol. It is noted that in specific embodiments of the present invention, glycerol could be substituted or replaced by other polyols such as glucose, fructose, sucrose, sorbitol, ethylene glycol, or polyethylene glycol; and agarose could be substituted or replaced by other hydrocolloids with the ability to thicken such as tragacanth gum, karaya gum, pectin, carrageenan, cellulose or modified cellulose or starch. In a preferred embodiment, the cold-adapted aquatic species is selected from a species of the genus Salmo or Oncorhynchus. Preferably, the cold-adapted aquatic species is selected from the group consisting of Salmo salar, Oncorhynchus nerka, Oncorhynchus tshawytscha, Oncorhynchus keta, Oncorhynchus kisutch, Oncorhynchus masou and Oncorhynchus gorbuscha. More preferably, the cold-adapted aquatic species is Salmo salar. It is noted that the above said amino acid composition of gelatin derived from cold-adapted aquatic species, is characterized by a repeating sequence of Gly-X-Y triplets, where X is mostly proline and Y is mostly hydroxyproline. Such gelatins are further characterized by having a proline and hydroxyproline content which is lower than that of gelatin isolated from mammalian species. Overall, amino acid composition of gelatin derived from cold-adapted aquatic species have lower concentrations of imino acids (proline and hydroxyproline) compared to mammalian gelatins, and warm-water fish gelatins (such as bigeye-tuna and tilapia) have a higher imino acid content that cold-water fish (such as cod, whiting and halibut) gelatins. The proline and hydroxyproline contents are approximately 30% for mammalian gelatins, 22 to 25% for warm-water fish gelatins (tilapia and Nile perch), and 17% for cold-water fish gelatin (these percentages are calculated based on the number of proline and hydroxyproline residues/1000 amino acid residues).

In a particular embodiment of the first aspect of the invention, the composition comprises 0.3 to 2% (w/v) gelatin derived from a cold-adapted aquatic species or a gelatin characterized by presenting a content of proline and hydroxyproline equal or less than 20%, preferably equal or less than 19%, 18%, 17%, 16% or 15% (these percentages are calculated based on the number of proline and hydroxyproline residues/1000 amino acid residues). In addition to, or alternatively, such amino acidic chain gelatin polymer is characterized by presenting 50 to 60 residues of hydroxyproline per 100 total amino acid residues and from 95 to 115 residues of proline per 1000 total amino acid residues.

The term “derived from a cold-adapted aquatic species” refers to any material which is derivable from biological material or from sequence information which has been obtained from a cold-adapted aquatic species. Therefore, any gelatin derived from a cold-adapted aquatic species also encompasses a gelatin which has been produced recombinantly with an amino acid sequence which is at least 75, 80, 85, 90, 95, 99 or 100% identical to a gelatin derived from a cold-adapted aquatic species as well as any gelatin extracted from tissue obtained from a cold-adapted aquatic species.

We have found that any material developed based on gelatin derived from a cold-adapted aquatic species which does not include any natural polymers derived from mammalian tissue minimizes or negates any risk of zoonosis. For example, because the composition or biomaterial of the present invention does not contain any bovine-derived material, there is less risk of infecting a patient with a transmissible spongiform encephalopathy. Further, a composition or biomaterial which does not comprise material derived from mammals can be used in countries where products containing material derived from certain mammals are prohibited for religious or cultural reasons.

Due to the rheological properties of gelatin derived from cold-adapted aquatic species, the material can be manipulated in a more controlled fashion than porcine or bovine gelatins which gel at ambient temperature. Therefore, compositions or biomaterials which comprise a gelatin derived from cold-adapted aquatic species will be more homogenous because the gelatin will remain aqueous during the manufacturing process.

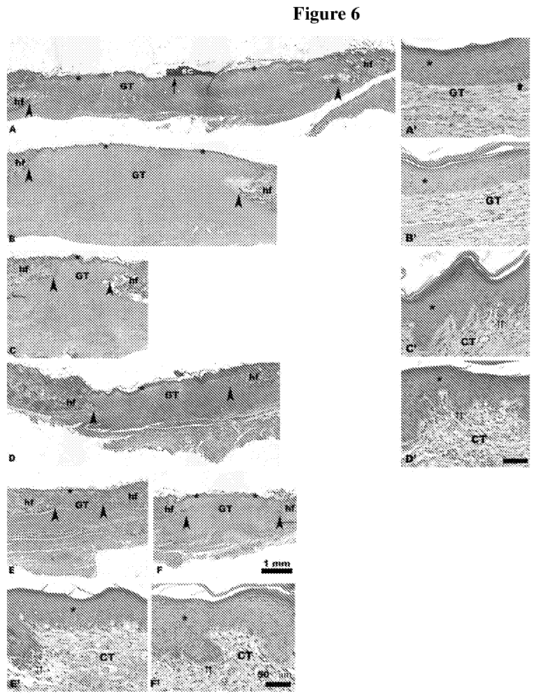

Further, the composition, pharmaceutical composition and biomaterial of the present invention were shown to be effective at improving the healing process in the Examples of the present disclosure. This is surprising considering the large phylogenetic distance between cold-adapted aquatic species and mammals More surprising was that the composition, pharmaceutical composition and biomaterial of the present invention induced the regeneration of hair follicles (see FIG. 6) whereas other implant systems were not able to regenerate a wound to the same extent (Woodroof, et al., 2015. “Evolution of a Biosynthetic Temporary Skin Substitute: A Preliminary Study” Eplasty, 15: e30; Weinstein-Oppenheimer, et al., 2010. “The effect of an autologous cellular gel-matrix integrated implant system on wound healing” J. Transl. Med., 8: 59).

Due to some of the reasons above, in a preferred embodiment, the composition does not comprise natural polymers and/or materials derived from pig and/or cow. Preferably, the composition does not comprise natural polymers and/or materials derived from mammals.

Chitosan is added to the composition to provide structural support and antimicrobial properties, agarose is added to the composition to provide structural support and glycerol is added as an excipient to improve the viscoelastic properties of the composition or biomaterial. In a preferred embodiment, the concentration of chitosan in the composition is 0.1 to 0.7% (w/v). In a preferred embodiment, the concentration of agarose in the composition is 0.05 to 0.3% (w/v). In a preferred embodiment, the concentration of glycerol in the composition is 0.01 to 0.2% (w/v). In a preferred embodiment, the concentration of chitosan in the composition is 0.1 to 0.7% (w/v) and the concentration of agarose in the composition is 0.05 to 0.3% (w/v). More preferably, the concentration of chitosan in the composition is 0.1 to 0.7% (w/v), the concentration of agarose in the composition is 0.05 to 0.3% (w/v) and the concentration of glycerol in the composition is 0.01 to 0.2% (w/v).

We have found that a ratio of 3:1:1 (gelatin:chitosan:agarose) provides a composition or biomaterial with good structural and mechanical properties. Therefore, in a preferred embodiment, the ratio of gelatin:chitosan:agarose in the composition is 3:1:1.

In a preferred embodiment, the composition is chemically crosslinked. Using a chemical crosslinker on the composition increases its mechanical stability. The composition may be crosslinked with any crosslinker known in the art using the information available in Bioconjugate Techniques, 3rd Edition (2013) by Greg T. Hermanson. In a preferred embodiment, the composition is crosslinked using one or more compounds which contain at least one chemical moiety selected from the group consisting of carbodiimide, N-hydroxysuccinimide (NHS), hydroxybenzotriazole, 1-hydroxy-7-azabenzotriazole, sulfo-NHS, imidoester, aldehyde, pyridyl disulfide, isothiocyanate, isocyanate, acyl azide, sulfonyl chloride, anhydride, fluorobenzene, epoxide, carbonate, fluorophenyl ester, hydrazide, alkoxyamine, maleimide and haloacetyl. Preferably, the composition is crosslinked using an NHS and a carbodiimide. More preferably, the composition is crosslinked using NHS and 1-ethyl-3-(3-dimethylaminopropyl)carbodiimide hydrochloride. In a preferred embodiment, the composition is not crosslinked with glutaraldehyde. Further manners of crosslinking such as by using enzymes, or further crosslinkers such as genipin or glutaraldehyde, as also included within the scope of the present invention.

In a preferred embodiment, the composition does not comprise hyaluronic acid.

In a second aspect, the present invention provides a pharmaceutical composition comprising the composition of the present invention and a pharmaceutically acceptable carrier or diluent.

As used herein, “pharmaceutically acceptable carrier” or “pharmaceutically acceptable diluent” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and, without limiting the scope of the present invention, include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone.

A pharmaceutical composition as described herein may also contain other substances. These substances include, but are not limited to, cryoprotectants, lyoprotectants, surfactants, bulking agents, anti-oxidants, and stabilizing agents.

The term “cryoprotectant” as used herein, includes agents which provide stability to the composition against freezing-induced stresses. Cryoprotectants may also offer protection during primary and secondary drying and long-term product storage. Non-limiting examples of cryoprotectants include sugars, such as sucrose, glucose, trehalose, mannitol, mannose, and lactose; polymers, such as dextran, hydroxyethyl starch and polyethylene glycol; surfactants, such as polysorbates (e.g., PS-20 or PS-80); and amino acids, such as glycine, arginine, leucine, and serine. A cryoprotectant exhibiting low toxicity in biological systems is generally used.

In one embodiment, a lyoprotectant is added to a pharmaceutical composition described herein. The term “lyoprotectant” as used herein, includes agents that provide stability to the composition during the freeze-drying or dehydration process (primary and secondary freeze-drying cycles), by providing an amorphous glassy matrix and by binding with the material's surface through hydrogen bonding, replacing the water molecules that are removed during the drying process. This helps to minimize product degradation during the lyophilization cycle, and improve the long-term product stability. Non-limiting examples of lyoprotectants include sugars, such as sucrose or trehalose; an amino acid, such as monosodium glutamate, non-crystalline glycine or histidine; a methylamine, such as betaine; a lyotropic salt, such as magnesium sulfate; a polyol, such as trihydric or higher sugar alcohols, e.g., glycerin, erythritol, glycerol, arabitol, xylitol, sorbitol, and mannitol; propylene glycol; polyethylene glycol; pluronics; and combinations thereof. The amount of lyoprotectant added to a pharmaceutical composition is generally an amount that does not lead to an unacceptable amount of degradation of the strain when the pharmaceutical composition is lyophilized.

In some embodiments, a bulking agent is included in the pharmaceutical composition. The term “bulking agent” as used herein, includes agents that provide the structure of the freeze-dried product without interacting directly with the pharmaceutical product. In addition to providing a pharmaceutically elegant cake, bulking agents may also impart useful qualities in regard to modifying the collapse temperature, providing freeze-thaw protection, and enhancing the composition stability over long-term storage. Non-limiting examples of bulking agents include mannitol, glycine, lactose, and sucrose. Bulking agents may be crystalline (such as glycine, mannitol, or sodium chloride) or amorphous (such as dextran, hydroxyethyl starch) and are generally used in formulations in an amount from 0.5% to 10%.

Other pharmaceutically acceptable carriers, excipients, or stabilizers, such as those described in Remington's Pharmaceutical Sciences 16th edition, Osol, A. Ed. (1980) may also be included in a pharmaceutical composition described herein, provided that they do not adversely affect the desired characteristics of the pharmaceutical composition. As used herein, “pharmaceutically acceptable carrier” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone.

### Process for Manufacturing a Biomaterial

In a third aspect, the present invention provides a process for manufacturing a biomaterial which comprises the steps of: a) mixing gelatin derived from a cold-adapted aquatic species, preferably from an aquatic species of the genus Salmo or Oncorhynchus, with chitosan, agarose and glycerol, wherein the final concentration of gelatin is 0.3 to 2% (w/v); b) drying the solution obtained in step (a); and

c) chemically crosslinking the mixture of step (b).

In a preferred embodiment, the cold-adapted aquatic species is selected from a group consisting of Salmo salar, Oncorhynchus nerka, Oncorhynchus tshawytscha, Oncorhynchus keta, Oncorhynchus kisutch, Oncorhynchus masou and Oncorhynchus gorbuscha. More preferably, the cold-adapted aquatic species is Salmo salar.

The mixture of step (b) may be crosslinked with any crosslinker known in the art using the information available in Bioconjugate Techniques, 3rd Edition (2013) by Greg T. Hermanson. In a preferred embodiment, the mixture of step (b) is crosslinked using one or more compounds which contain at least one chemical moiety selected from the group consisting of carbodiimide, N-hydroxysuccinimide (NHS), hydroxybenzotriazole, 1-hydroxy-7-azabenzotriazole, sulfo-NHS, imidoester, aldehyde, pyridyl disulfide, isothiocyanate, isocyanate, acyl azide, sulfonyl chloride, anhydride, fluorobenzene, epoxide, carbonate, fluorophenyl ester, hydrazide, alkoxyamine, maleimide and haloacetyl. Preferably, the mixture of step (b) is crosslinked using an NHS and a carbodiimide. More preferably, the mixture of step (b) is crosslinked using NHS and 1-ethyl-3-(3-dimethylaminopropyl)carbodiimide hydrochloride. In a preferred embodiment, the mixture of step (b) is not crosslinked with glutaraldehyde.

In a preferred embodiment, the biomaterial obtained after step (c) is sterilized using radiation. Preferably, the biomaterial is irradiated using gamma radiation. We have found that the mechanical properties of the biomaterial are unaffected by gamma radiation. In a preferred embodiment, the biomaterial obtained after step (c) is sterilized using 20 to 50 kGy of gamma radiation, preferably 25 kGy.

In a preferred embodiment, the concentration of chitosan in step (a) is 0.1 to 0.7% (w/v). In a preferred embodiment, the concentration of agarose in step (a) is 0.05 to 0.3% (w/v). In a preferred embodiment, the concentration of glycerol in step (a) is 0.01 to 0.2% (w/v). In a preferred embodiment, the concentration of chitosan in step (a) is 0.1 to 0.7% (w/v) and the concentration of agarose in step (a) is 0.05 to 0.3% (w/v). More preferably, the concentration of chitosan in step (a) is 0.1 to 0.7% (w/v), the concentration of agarose in step (a) is 0.05 to 0.3% (w/v) and the concentration of glycerol in step (a) is 0.01 to 0.2% (w/v). In a preferred embodiment, the ratio of gelatin:chitosan:agarose in step (a) is 3:1:1.

In a preferred embodiment, the solution is dried by lyophilizing the product obtained in step (a) of the process.

In a preferred embodiment, the process does not comprise the use of natural polymers and/or materials derived from pig and/or cow. Preferably, the process does not comprise the use of natural polymers and/or materials derived from mammals.

In preferred embodiment, the process does not comprise the use of hyaluronic acid.

In a preferred embodiment, the process comprises the steps of:
a) mixing gelatin derived from a cold-adapted aquatic species, preferably from an aquatic species of the genus Salmo or Oncorhynchus, with chitosan, agarose and glycerol, wherein the final concentration of gelatin is 0.3 to 2% (w/v);
b) drying, preferably lyophilizing, the mixture of step (a);
c) rehydrating the lyophilisate of step (b);
d) chemically crosslinking the solution of step (c); and
e) drying, preferably lyophilizing, the product obtained in step (d).

In a preferred embodiment, the cold-adapted aquatic species is selected from a group consisting of Salmo salar, Oncorhynchus nerka, Oncorhynchus tshawytscha, Oncorhynchus keta, Oncorhynchus kisutch, Oncorhynchus masou and Oncorhynchus gorbuscha. More preferably, the cold-adapted aquatic species is Salmo salar.

The solution of step (c) may be crosslinked with any crosslinker known in the art using the information available in Bioconjugate Techniques, 3rd Edition (2013) by Greg T. Hermanson. In a preferred embodiment, solution of step (c) is crosslinked using one or more compounds which contain at least one chemical moiety selected from the group consisting of carbodiimide, N-hydroxysuccinimide (NHS), hydroxybenzotriazole, 1-hydroxy-7-azabenzotriazole, sulfo-NHS, imidoester, aldehyde, pyridyl disulfide, isothiocyanate, isocyanate, acyl azide, sulfonyl chloride, anhydride, fluorobenzene, epoxide, carbonate, fluorophenyl ester, hydrazide, alkoxyamine, maleimide and haloacetyl. Preferably, the solution of step (c) is crosslinked using an NHS and a carbodiimide. More preferably, the solution of step (c) is crosslinked using NHS and 1-ethyl-3-(3-dimethylaminopropyl)carbodiimide hydrochloride. In a preferred embodiment, the solution of step (c) is not crosslinked with glutaraldehyde.

In a preferred embodiment, the biomaterial obtained in step (e) is sterilized using radiation. Preferably, the biomaterial is irradiated using gamma radiation. We have found that the mechanical properties of the biomaterial are unaffected by gamma radiation. In a preferred embodiment, the biomaterial obtained in step (e) is sterilized using 20 to 50 kGy of gamma radiation, preferably 25 kGy.

In a preferred embodiment, the concentration of chitosan in step (a) is 0.1 to 0.7% (w/v). In a preferred embodiment, the concentration of agarose in step (a) is 0.05 to 0.3% (w/v). In a preferred embodiment, the concentration of glycerol in step (a) is 0.01 to 0.2% (w/v). In a preferred embodiment, the concentration of chitosan in step (a) is 0.1 to 0.7% (w/v) and the concentration of agarose in step (a) is 0.05 to 0.3% (w/v). More preferably, the concentration of chitosan in step (a) is 0.1 to 0.7% (w/v), the concentration of agarose in step (a) is 0.05 to 0.3% (w/v) and the concentration of glycerol in step (a) is 0.01 to 0.2% (w/v). In a preferred embodiment, the ratio of gelatin:chitosan:agarose in step (a) is 3:1:1.

In a preferred embodiment, the process does not comprise the use of natural polymers and/or materials derived from pig and/or cow. Preferably, the process does not comprise the use of natural polymers and/or materials derived from mammals.

In preferred embodiment, the process does not comprise the use of hyaluronic acid.

### Biomaterial

In a fourth aspect, the present invention provides a biomaterial obtained or obtainable through any of the processes of the present invention which were described previously.

In a preferred embodiment the biomaterial comprises chemically crosslinked gelatin derived from a cold-adapted aquatic species; (ii) chitosan; (iii) agarose; and (iv) glycerol, wherein the biomaterial is a dried material. In a preferred embodiment, the biomaterial is sterile.

The biomaterial may be crosslinked with any crosslinker known in the art using the information available in Bioconjugate Techniques, 3rd Edition (2013) by Greg T. Hermanson. In a preferred embodiment, the biomaterial is crosslinked using one or more compounds which contain at least one chemical moiety selected from the group consisting of carbodiimide, N-hydroxysuccinimide (NHS), hydroxybenzotriazole, 1-hydroxy-7-azabenzotriazole, sulfo-NHS, imidoester, aldehyde, pyridyl disulfide, isothiocyanate, isocyanate, acyl azide, sulfonyl chloride, anhydride, fluorobenzene, epoxide, carbonate, fluorophenyl ester, hydrazide, alkoxyamine, maleimide and haloacetyl. Preferably, the biomaterial is crosslinked using an NHS and a carbodiimide. More preferably, the biomaterial is crosslinked using NHS and 1-ethyl-3-(3-dimethylaminopropyl)carbodiimide hydrochloride. In a preferred embodiment, the biomaterial is not crosslinked with glutaraldehyde.

In a preferred embodiment, the ratio of gelatin:chitosan:in the biomaterial is 3:1:1. In a preferred embodiment, the biomaterial is biologically active, biocompatible and biodegradable.

In a preferred embodiment, the biomaterial does not comprise natural polymers and/or materials derived from pig and/or cow. Preferably, the biomaterial does not comprise natural polymers and/or materials derived from mammals.

In a preferred embodiment, the biomaterial does not comprise hyaluronic acid.

Kit

In a fifth aspect, the present invention provides a kit comprising (i) gelatin derived from a cold-adapted aquatic species, preferably from an aquatic species of the genus Salmo or Oncorhynchus; (ii) chitosan; (iii) agarose; and (iv) glycerol. Preferably, the cold-adapted aquatic species is selected from the group consisting of Salmo salar, Oncorhynchus nerka, Oncorhynchus tshawytscha, Oncorhynchus keta, Oncorhynchus kisutch, Oncorhynchus masou and Oncorhynchus gorbuscha. More preferably, the cold-adapted aquatic species is Salmo salar. In a preferred embodiment, the kit does not comprise natural polymers and/or materials derived from pig and/or cow. Preferably, the kit does not comprise natural polymers and/or materials derived from mammals.

In a preferred embodiment, the kit further comprises at least one crosslinker. Preferably the crosslinker is selected from one or more compounds which contain at least one chemical moiety selected from the group consisting of carbodiimide, N-hydroxysuccinimide (NHS), hydroxybenzotriazole, 1-hydroxy-7-azabenzotriazole, sulfo-NHS, imidoester, aldehyde, pyridyl disulfide, isothiocyanate, isocyanate, acyl azide, sulfonyl chloride, anhydride, fluorobenzene, epoxide, carbonate, fluorophenyl ester, hydrazide, alkoxyamine, maleimide and haloacetyl. Preferably, the crosslinker is selected from the group consisting of an NHS and a carbodiimide. More preferably, the kit further comprises NHS and 1-ethyl-3-(3-dimethylaminopropyl)carbodiimide hydrochloride. In a preferred embodiment, the kit does not comprise glutaraldehyde.

In a preferred embodiment, the kit may further comprise instructions for producing biomaterials in accordance with any of the processes of the present invention.

### Uses of the Composition, Biomaterial or Kit.

In a sixth aspect, the present invention provides the use of the composition of the present invention, the biomaterial of the present invention, the pharmaceutical composition of the present invention or the kit of the present invention for the production of scaffolds, dressings, beads, engineered tissues, devices or micro-devices suitable for therapeutic or diagnostic purposes.

The term “therapeutic purpose”, as used in the present application, refers to the use of scaffolds, dressings, beads, engineered tissues, devices or micro-devices with the intent to cure and/or alleviate a disease and/or symptoms with the goal of remediating the health problem. The term “therapeutic purpose” includes preventive and curative purposes, since both are directed to the maintenance and/or reestablishment of the health of an individual or animal.

The term “diagnostic purpose”, as used in the present application, refers to the use of scaffolds, dressings, beads, engineered tissues, devices or micro-devices with the intent to identify and/or evaluate a disease and/or the origins of one or more symptoms.

The term “scaffold” refers to a structure which serves as a support for other materials and/or tissue. For example, the scaffold of the present invention may be used to grow organs from tissue culture.

The term “dressing” refers to a piece of material used to cover and protect a wound. In a preferred embodiment, the dressing is used for the treatment of a wound. Preferably, the wound is epidermal, dermal or hypodermal.

The term “bead” refers to a micro- or nanoparticle which is usually spherical or somewhat spherical in shape which can be functionalized. For example, the composition of the present invention could be used to make beads which are then functionalized with antibodies which bind to a specific target of interest. The beads could therefore be used for diagnostic purposes.

The term “engineered tissue” refers to a live tissue obtained using a combination of cells, engineering and materials methods, and suitable biochemical and physicochemical factors to improve or replace biological tissues. For example, the biomaterial may be used as a scaffold to grow a heart valve which can then be transplanted in a patient who suffers from aortic regurgitation.

In a preferred embodiment, the composition of the present invention, the biomaterial of the present invention, the pharmaceutical composition of the present invention or the kit of the present invention is used for the production of beads, devices or micro-devices suitable for diagnostic purposes.

In a seventh aspect, the present invention provides the use of the composition of the present invention, the biomaterial of the present invention, the pharmaceutical composition of the present invention or the kit of the present invention for tissue engineering.

The term “tissue engineering” refers to the use of a combination of cells, engineering and materials methods, and suitable biochemical and physicochemical factors to improve or replace biological tissues. In a preferred embodiment, the composition of the present invention, the biomaterial of the present invention or the kit of the present invention is used to produce a scaffold for tissue engineering.

In an eighth aspect, the present invention provides the use of the composition of the present invention, the biomaterial of the present invention, the pharmaceutical composition of the present invention or the kit of the present invention for the production of a dressing for topical administration.

### EXAMPLES

### Example 1: Extraction of Gelatin from Salmon

The gelatin used in the present examples was extracted from salmon skin. Specifically, the skin was obtained from Salmo salar. The skins were cleaned by removing the scales and any residual muscular tissue. Then the skin was cut into small pieces and submerged in a solution of 0.1 M NaOH at a 1:6 ratio (skin:solution) for 1 hour and 10° C. under constant agitation.

The pieces of skin were washed with distilled water and then submerged again in a solution of 0.1 M NaOH at a 1:6 ratio (skin:solution) for 1 hour and 10° C. under constant agitation. The pieces of skin were washed again with distilled water and then submerged in a solution of 0.05 M acetic acid (CH₃COOH) at a 1:6 ratio (skin:solution) for 1 hour and 10° C. under constant agitation. The pieces of skin were washed again with distilled water and then submerged in distilled water at a 1:6 ratio (skin:solution). The pH of the distilled water was then adjusted to 4.0 using acetic acid and then the submerged skin pieces were incubated for 3.5 hours at 60° C. under constant agitation. The temperature and pH were monitored throughout the incubation period.

The pieces of skin were then removed, and the supernatant was then filtered through a 0.22 μm filter. The filtered suspension was then dried at 55° C. for 24-48 hours and the resultant solid product was ground and stored at 4° C. prior to use.

### Example 2: Production of the Biomaterial

STAGE 1: The gelatin obtained in Example 1, agarose (Sigma-Aldrich) and glycerol (Merck) were dissolved into distilled water and chitosan (Quitoquímica) was dissolved into 1% (v/v) acetic acid. The dissolved components were mixed to the final concentrations disclosed in Table 1. The resultant solution was then lyophilized.

STAGE 2: 1 g of the resultant lyophilisate was immersed for 2 h into 10 ml of a solution comprising 30 mM of 1-ethyl-3-(3-dimethylaminopropyl)carbodiimide (Sigma-Aldrich) and 8 mM of N-hydroxysuccinimide (Sigma-Aldrich), using ethanol 90% v/v as solvent and MES as buffer (50 mM). Cross-linking resulted in the formation of a porous insoluble material. The resultant composition was lyophilized to obtain a dry insoluble matrix (the biomaterial).

STAGE 3: The biomaterial was then sterilized using 25 kGy of gamma radiation.

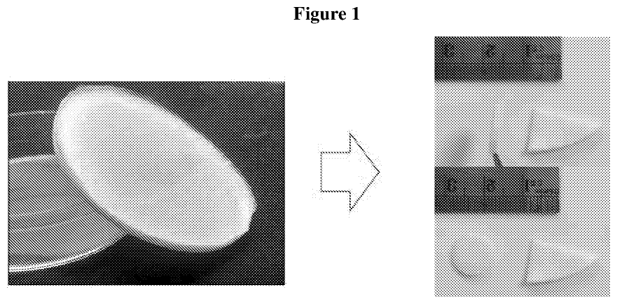

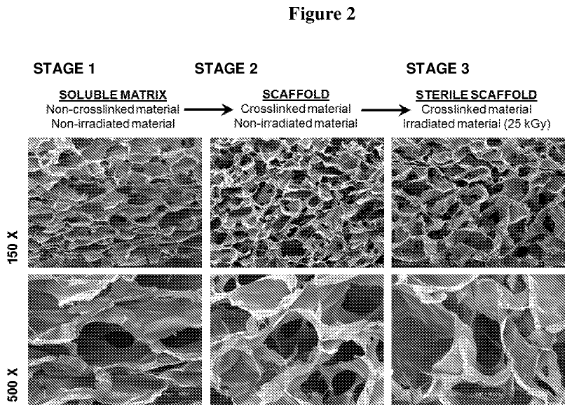

FIG. 1 shows the resultant homogenous sponge-like biomaterial obtained.

TABLE 1
Contents of the Composition and the resultant Biomaterial

| | Concentration in solution | Biomaterial composition |
|---|---|---|
| Polymer | (%) | (%) |
| Gelatin | 0.6 | 54.5 |
| Chitosan | 0.2 | 18.2 |
| Agarose | 0.2 | 18.2 |
| Glycerol | 0.1 | 9.1 |

### Example 3: Scanning Electron Microscopy (SEM) of the Biomaterial

The materials obtained at the different stages of production of the biomaterial of Example 2 were imaged using a Scanning Electron Microscope Carl Zeiss SEM (EVO MA 10, Germany Samples were coated with gold and observed at 150× and 500×.

As can be seen in FIG. 2, the stage 1 material, which is obtained after lyophilizing the initial mixture of components, produces a porous material. The stage 2 material, i.e. after crosslinking, has a more organized and stable porous structure. The porous structure of the stage 2 material appears to be unaffected by gamma radiation as can be seen in the images of the stage 3 material. The images obtained for the stage 2 and stage 3 material indicate that the pores are the appropriate size for cell culture.

### Example 4: Mechanical Properties of the Biomaterial

The materials obtained at the three stages of production were tested using an texture analyzer (TA.XT Plus Stable Micro Systems, UK). The results obtained are outlined in Table 2. Briefly, crosslinking increased the Young's modulus without resulting in a more fragile material.

TABLE 2
Mechanical properties of the materials

| | | Young's | | |
|---|---|---|---|---|
| | MC | Modulus | Stress at break | Strain at break |
| | (%) | (Pa) | (Pa) | (%) |
| STAGE 1 | 13.7 ± 0.5 | 130 ± 18 | 475 ± 35 | 3.28 ± 0.48 |
| STAGE 2 | 10.6 ± 0.1 | 150 ± 17 | 317 ± 18 | 2.48 ± 0.99 |
| STAGE 3 | 10.4 ± 0.1 | 170 ± 20 | 462 ± 24 | 3.29 ± 0.01 |

### Example 5: Hydrophilicity of the Biomaterial

The hydrophilicity of the biomaterial was measured dynamically by a Dynamic Vapor sorption (DVS) (Intrinsic High Mass, Surface Measurement Systems, UK) was used. Briefly, 10 mg of sample was loaded into the DVS sample cell. The experimental protocol used was as follow:

drying by dry nitrogen flow at 0.0 RH until an equilibrium was reached. The samples were then subjected to a sorption cycle using 0.1 (RH/100) increments between 0 and 0.8 (RH/100). Equilibrium mass, at each RH, was determined when dm/dt=0.002% min⁻¹. Moisture sorption isotherms were described for each powder blend by fitting the equilibrium moisture sorption data in the RH range between 0.0 and 0.8 (RH/100) with the GAB isotherm model (Guggenheim, 1966). The moisture content data were expressed in dry basis.

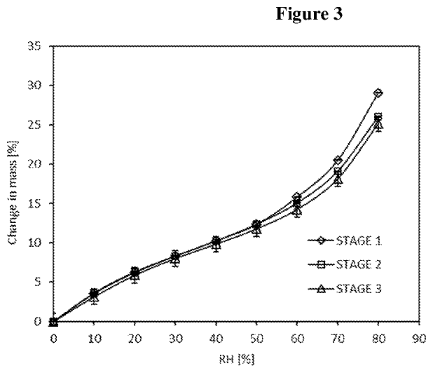

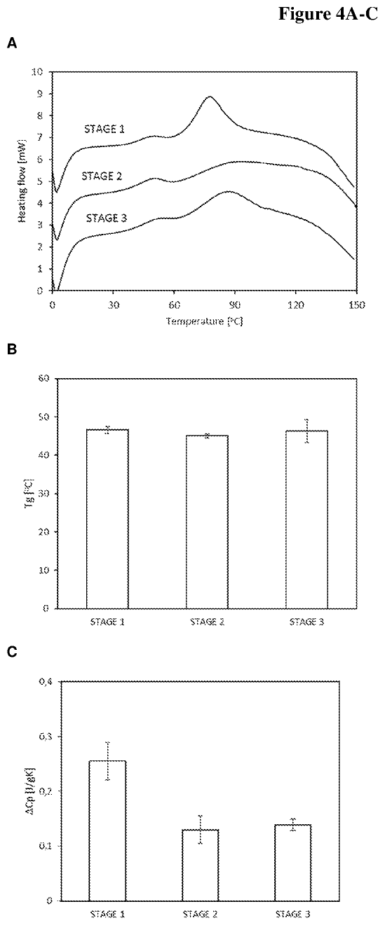

As can be seen in FIG. 3, there is little change in the interaction between the material after crosslinking or irradiation with water. The relative humidity causes a similar increase in mass in the three samples and the sigmoidal sorption profile for typical hydrophilic composites remains.

Similar results were obtained using the GAB (Guggenhaim, Anderson and de Boer, (Guggenheim, 1966).) equation:

$$M = \frac{{m}_{0} {C}_{GAB} K {a}_{w}}{( 1 - {a}_{w} ) ( 1 - K {a}_{w} + {C}_{GAB} K {a}_{w} )}$$

Where, M is water content; m₀ is moisture content needed to cover the entire surface with a unimolecular layer; C_{GAB} is constant associated with the monolayer enthalpy of sorption; K is factor correcting properties of enthalpy of sorption of the multilayer molecules with respect to the bulk liquid; aw is water activity.
Guggenheim, E., 1966. Applications of statistical mechanics. Oxford, Clarendon Press.

TABLE 3
Parameters calculated using the GAB equation

| | | | | Error |
|---|---|---|---|---|
| | m₀ | C_{GAB} | Ka_{w} | (%) |
| STAGE 1 | 7.7 | 7.2 | 0.93 | 1.1 |
| STAGE 2 | 7.5 | 5.9 | 0.90 | 3.3 |
| STAGE 3 | 7.1 | 7.2 | 0.90 | 2.0 |

### Example 6: Thermal Properties of the Biomaterial

Differential scanning calorimetry (DSC) was used to study the thermal properties of the materials obtained in each stage of production. Specifically, a DSC 1 STAR System (Mettler-Toledo, Switzerland) was used. The material was analyzed as received. Approximately 20 mg of film was loaded into aluminum pan (40 μl) and then hermetically sealed. An empty aluminum pan was used as a reference. The samples were scanned at a rate of 10° C./min from 0 to 150° C. Melting temperature and enthalpy calibration was carried out using indium as the standard material (Tm=156.6° C. ΔHm=28.55 J/g). The thermal parameters were obtained by using the instrument software (STARe Software, Mettler-Toledo).

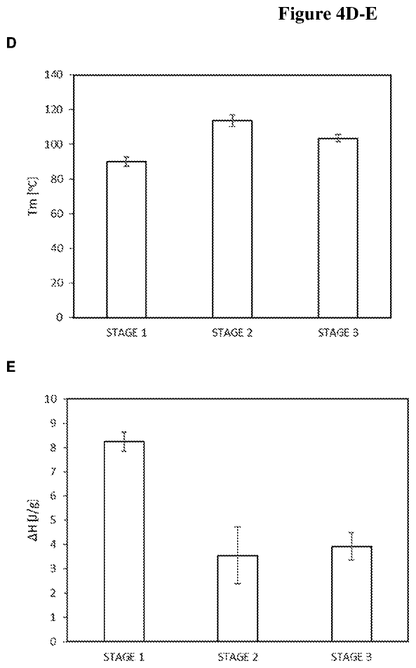

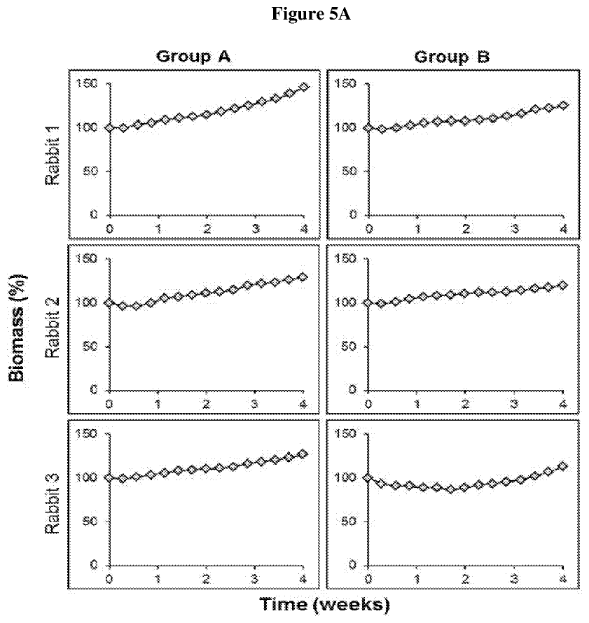

FIG. 4A shows exemplary melting profiles obtained for the three materials.

FIG. 4B shows that the Tg (glass transition temperature) was similar for the three materials. However, crosslinking was shown to decrease the heat capacity (Cp) of the material (FIG. 4C). The decrease in Cp is probably due to a restriction in the molecular movement of the material caused by the covalent crosslinks.

FIG. 4D shows that the Tm (melting temperature) increases after crosslinking. Further, FIG. 4E shows that the change in enthalpy (AH) decreases due to crosslinking.

### Example 7: In Vivo Studies Using the Biomaterial

Two groups of rabbits of the species Oryctolagus cuniculus, each comprising three brothers of the same litter, were wounded on the dorsolumbar. The wound was circular and 4 cm in diameter. A circular dressing made of the sterilized biomaterial produced in Example 2 was placed on the wound. The rabbits were then left to recover for 4 hours in individual cages. The rabbits were monitored for growth, physiological changes, cicatrization and superficial anomalies every 48 hours.

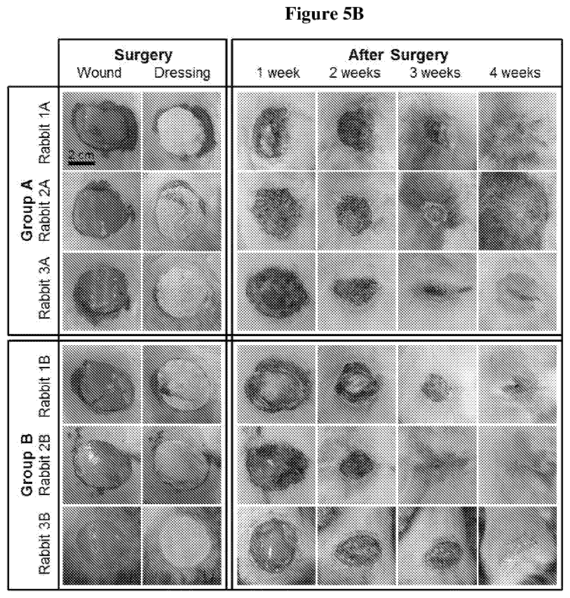

The rabbits were lightly dehydrated 3 days after surgery but recovered on the fourth day. Their body temperature was also elevated by 0.4 to 0.7° C. two days after surgery but normalized on the third day. The rabbits recovered well with the dressing as can be seen in their growth (FIG. 5A).

Cicatrization occurred without significant physiological changes and in the absence of any significant inflammatory response (FIG. 5B). The biomaterial did not present any clinical safety issues 30 days after its application.

In vivo pre-clinical trials have demonstrated an excellent adherence of the biomaterial to the wound in comparison to other similar products. Further, the biomaterial is incorporated more quickly by the host in comparison to other similar products, which favors rapid skin regeneration. These properties might be related to the biomaterial's unique viscoelastic profile which allows for a greater degree of intermolecular movement. This might make the structure of the biomaterial easier to remodel for the host cells. Further, the gelatin derived from salmon skin comprises a larger amount of glycine and aspartic acid in comparison to bovine gelatin. Therefore, salmon gelatin might contain more RGD motifs which stimulate cellular adhesion which is important for tissue regeneration.

TABLE 4
Comparison of the arginine, glycine and aspartic acid content
of salmon-derived gelatin and bovine-derived gelatin

| | Salmon-derived gelatin | Bovine-derived gelatin |
|---|---|---|
| Amino acid | (% p/p) | (% p/p) |
| Arginine | 5.03 | 8.89 |
| Glycine | 33.75 | 23.5 |
| Aspartic acid | 4.99 | 4.64 |
| TOTAL | 43.77 | 37.03 |

### Example 8: Histological Analysis of Cicatrized Skin

Rabbits were anesthetized with ketamine/xylazine. A selected dorsal area was shaved and disinfected. Then, a full-thickness excision wound was performed in each animal at the paravertebral skin, which was covered with the material.

Biopsy of the complete skin was taken for histological analysis. The biopsies were fixed in Bouin's solution, processed with standard histological techniques and stained with Hematoxylin/Erythrosine B-Orange G/Methyl blue.

The rabbits implanted with the dressing exhibited complete epithelialization. Areas that were exposed to the biomaterial showed similar characteristics to that of completely cicatrized tissues which had not been exposed to a biomaterial. Further, there were no signs of rejection (FIG. 6).

1. A composition comprising:
i) 0.3 to 2% (w/v) gelatin derived from a cold-adapted aquatic species, preferably from an aquatic species of the genus Salmo or Oncorhynchu, or characterized by having an amino acidic chain comprising 50 to 60 residues of hydroxyproline per 100 total amino acid residues and from 95 to 115 residues of proline per 1000 total amino acid residues;
ii) chitosan;
iii) agarose; and
iv) glycerol.

2. The composition according to claim 1, wherein the concentration of chitosan is 0.1 to 0.7% (w/v).

3. The composition according to any one of the preceding claims, wherein the concentration of agarose is 0.05 to 0.3% (w/v).

4. The composition according to any one of the preceding claims, wherein the composition is chemically crosslinked.

5. The composition according to claim 4, wherein the composition is crosslinked using a carbodiimide and an N-hydroxysuccinimide.

6. A process for manufacturing a biomaterial which comprises the steps of:
a) mixing gelatin derived from a cold-adapted aquatic species, preferably from an aquatic species of the genus Salmo or Oncorhynchus, with chitosan, agarose and glycerol, wherein the final concentration of gelatin is 0.3 to 2% (w/v);
b) drying the solution obtained in step (a); and
c) chemically crosslinking the mixture of step (b).

7. The process according to claim 6, wherein the process further comprises a step where the dried biomaterial obtained after step (c) is sterilized using radiation.

8. The process according to claim 7, wherein the biomaterial is irradiated using gamma radiation.

9. The process according to any one of claims 6-8, wherein the concentration of chitosan in step (a) is 0.1 to 0.7% (w/v).

10. The process according to any one of claims 6-9, wherein the concentration of agarose in step (a) is 0.05 to 0.3% (w/v).

11. The process according to any one of claims 6-10, wherein the solution is dried by lyophilizing the product obtained in step (a).

12. A biomaterial obtained or obtainable through the process according to any one of claims 6-11.

13. A pharmaceutical composition comprising the composition according to any one of claims 1-5 and a pharmaceutically acceptable carrier or diluent.

14. A kit comprising:
(i) gelatin derived from a cold-adapted aquatic species, preferably from an aquatic species of the genus Salmo or Oncorhynchus;
(ii) chitosan;
(iii) agarose; and
(iv) glycerol.

15. Use of the composition according to any one of claims 1-5, the biomaterial according to claim 12, the pharmaceutical composition according to claim 13 or the kit according to claim 14 for the production of scaffolds, dressings, beads, engineered tissues, devices or micro-devices suitable for therapeutic or diagnostics purposes.

16. Use of the composition according to any one of claims 1-5, the biomaterial according to claim 12, the pharmaceutical composition according to claim 13 or the kit according to claim 14 for tissue engineering.

17. Use of the composition according to any one of claims 1-5, the biomaterial according to claim 12, the pharmaceutical composition according to claim 13 or the kit according to claim 14 for the production of a dressing for topical administration.
